(19) United States
(12) Patent Application Publication
(10) Pub. No.: US 2024/0159660 A1
(43) Pub. Date: May 16, 2024

(54) LIGHT CRUDE OIL FLUID IDENTIFICATION WITHIN AN OBM DRILLING FLUID BASE/FILTRATE

(71) Applicant: Baker Hughes Oilfield Operations LLC, Houston, TX (US)

(72) Inventors: Carlos Toro, Bogota (CO); Sebastian Mesa, Bogota (CO); Camilo Puentes, Bogota (CO); Randy Van Der Ree, Maracaibo (VE); Dorianne Castillo, Humble, TX (US); Daniela Serrano, Bogota (CO)

(73) Assignee: Baker Hughes Oilfield Operations LLC, Houston, TX (US)

(21) Appl. No.: 17/985,092

(22) Filed: Nov. 10, 2022

(51) Int. Cl. G01N 21/33; G01N 33/28

(52) U.S. Cl. CPC G01N 21/33; G01N 33/2823

(57) ABSTRACT

A method for determining whether crude oil has been produced from a hydrocarbon reservoir wellbore that has been drilled using an oil-based mud (OBM). The method includes proving the OBM which contains a tracer, filtering the OBM to obtain an OBM filtrate comprising the tracer, determining a baseline absorbance value for the OBM filtrate containing the tracer, determining a baseline absorbance value for the crude oil, introducing the OBM filtrate into a hydrocarbon reservoir wellbore, conducting a wellbore operation with the OBM, producing a sample from the well, measuring a measured absorbance value for the sample, and comparing the measured absorbance value with the baseline absorbance values for the OBM filtrate and the baseline absorbance value for the crude oil to determine whether crude oil is present in the sample.

### TECHNICAL FIELD

The present invention relates to a method for determining whether crude oil has been produced from a well that has been drilled using a drilling fluid, and more particularly relates to a method for determining whether crude oil has been produced from a well that has been drilled using an oil-based drilling fluid (OBM) containing tracers.

### BACKGROUND

Drilling fluids used in the drilling of subterranean oil and gas wells along with other drilling fluid applications and drilling procedures are known. In rotary drilling, there are a variety of functions and characteristics that are expected of drilling fluids, also known as drilling muds, or simply “muds”. The drilling fluid should carry cuttings from beneath the bit, transport them through the annulus, and allow their separation at the surface, while the rotary bit is cooled and cleaned. A drilling mud is also intended to reduce friction between the drill string and the sides of the hole, while maintaining the stability of uncased sections of the borehole.

The drilling fluid is formulated to prevent unwanted influxes of formation fluids from permeable rocks penetrated. The drilling fluid is also often formulated to form a thin, low permeability filter cake which temporarily seals pores, other openings and formations penetrated by the bit. The drilling fluid may also be used to collect and interpret information available from drill cuttings, cores, and electrical logs. It will be appreciated that as defined herein, the term “drilling fluid” also encompasses “drill-in fluids” and “completion fluids”.

Drilling fluids are typically classified according to their base fluid. In water-based muds (WBMs), solid particles are suspended in water or brine. Oil can be emulsified in the water. Nonetheless, the water is the continuous phase. Oil-based muds (OBMs) are the opposite or inverse. Solid particles are suspended in oil, and water or brine is emulsified in the oil and therefore the oil is the continuous phase. Oil-based muds which are water-in-oil macroemulsions are also called invert emulsions. The oil in oil-based (invert emulsion) mud can consist of any oil that may include diesel, mineral oil, esters, or alpha olefins. Brine-based drilling fluids, of course are a water-based mud in which the aqueous component is brine. It is apparent to those selecting or using a drilling fluid for oil and/or gas exploration that an essential component of a selected fluid is that it be properly balanced to achieve the necessary characteristics for the specific end application.

Drilling fluids have a number of tasks or functions to perform simultaneously. One specific function of the drilling fluid is to form a filter cake to control the filtrate invasion into the formation. Filter cakes are the residue deposited on a permeable medium, such as a formation surface when a slurry or suspension, such as a drilling fluid, is circulated within the wellbore where the pressure is overbalanced. Filtrate is the liquid that passes through the medium, leaving the filter cake on the medium. Filter cake properties, such as cake thickness, toughness, slickness, and permeability are important because the cake that forms on permeable zones in a wellbore can cause stuck pipe and other drilling problems. Reduced hydrocarbon production can result from reservoir or skin damage when a poor filter cake allows deep filtrate invasion.

OBMs are normally used in complex situations, such as high pressure and high temperature (HPHT) environments. These OBMs allow improving the torque and drag parameters, and reduce the friction between the drill string and the formation. OBMs improve stability in shale formations, generating the least cavitation effect. One disadvantage of using an OBM is the identification of hydrocarbons in permeable and porous zones due to their chemical composition having very similar characteristics in terms of fluorescence and light absorbance of the crude oil that is produced. This effect limits the use of conventional log tools based on fluorescence and absorbance principles using logging tools including, but not necessarily limited to, Wireline, Measurement While Drilling (MWD), Logging While Drilling (LWD), and limits the ability of the operator to estimate reserves and prospects of the producing formations.

It would be desirable if methods could be devised to aid and improve the ability to determine when crude oil is produced together with OBM and/or OBM filtrate using a conventional downhole logging tool.

### SUMMARY

There is provided, in one form, a method for determining whether crude oil has been produced from a hydrocarbon reservoir wellbore that has been drilled using an oil-based mud (OBM), where the method includes providing the OBM, where the OBM includes a chemical tracer, filtering the OBM to obtain an OBM filtrate comprising the tracer, determining a baseline absorbance value for the OBM filtrate containing the chemical tracer, determining a baseline absorbance value for the crude oil, introducing the OBM filtrate into a hydrocarbon reservoir wellbore, conducting a wellbore operation with the OBM, producing a sample from the well, measuring a measured absorbance value of the sample, and comparing the measured absorbance value with the baseline absorbance values for the OBM filtrate and the baseline absorbance value for the crude oil to determine whether crude oil is present in the sample.

There is further provided in another non-limiting embodiment an oil-based mud (OBM) composition that includes an oil-based mud filtrate, and a tracer.

In a different non-restrictive version, there is provided a sample composition that contains crude oil produced from a hydrocarbon reservoir, an oil-based mud (OBM) filtrate, and a tracer.

### BRIEF DESCRIPTION OF THE DRAWINGS

FIG. 1 is a chart of the UV base line absorbance spectrum for a Colombian crude oil;

FIG. 2 is a chart of the UV base line absorbance spectrum for Fuel Oil #4;

FIG. 3 is a chart of the UV absorbance spectrum for the Colombian crude oil with Fuel Oil #4, in a ratio of 25% to 75%, and 2500 ppm green dye (Test #1);

FIG. 4 is a chart of the UV absorbance spectrum for the Colombian crude oil with Fuel Oil #4, in a ratio of 50% to 50%, and 2500 ppm green dye (Test #2);

FIG. 5 is a chart of the UV absorbance spectrum for the Colombian crude oil with Fuel Oil #4, in a ratio of 75% to 25%, and 2500 ppm green dye (Test #3);

FIG. 6 is a chart of the UV base line absorbance spectrum for 100% filtrate with 2500 ppm green dye;

FIG. 7 is a chart of the UV absorbance spectrum for the Colombian crude oil and filtrate, in a ratio of 25% to 75%, with 2500 ppm green dye (Test #4);

FIG. 8 is a chart of the UV absorbance spectrum for the Colombian crude oil and filtrate, in a ratio of 50% to 50%, with 2500 ppm green dye (Test #5);

FIG. 9 is a chart of the UV absorbance spectrum for the Colombian crude oil and filtrate, in a ratio of 75% to 25%, with 2500 ppm green dye (Test #6);

FIG. 10 is a chart of the UV base line absorbance spectrum for 100% filtrate with 2500 ppm red dye;

FIG. 11 is a chart of the UV absorbance spectrum for the Colombian crude oil with filtrate, in a ratio of 25% to 75%, with 2500 ppm red dye (Test #7);

FIG. 12 is a chart of the UV absorbance spectrum for the Colombian crude oil with filtrate, in a ratio of 50% to 50%, with 2500 ppm red dye (Test #8); and

FIG. 13 is a chart of the UV absorbance spectrum for the Colombian crude oil with filtrate, in a ratio of 75% to 25%, with 2500 ppm red dye (Test #9).

### DETAILED DESCRIPTION

A method has been discovered that uses chemical tracers in an oil-based drilling fluid (OBM) in a way which Wire-Line, MWD, and LWD logging tools with optical, density, and viscosity sensors can measure in-situ fluid properties in real time to determine if crude oil is being produced. The method is not necessarily limited to the logging tools mentioned above. In other words, the method determines the presence of chemical tracers in oil-based drilling systems and how to use Wireline, MWD, and LWD tools with optical sensors, along with sensors for density and viscosity for real-time, in-situ fluid property measurements for differentiating the flow of hydrocarbons from crude oil, filtrates, and blends thereof through the tools, which facilitates the characterization of fluids and quantification of hydrocarbon reserves.

Typically, in the drilling stage of a well, the filtrate invades the formation with an overbalancing effect, generating a mixture between the crude oil and the OBM or the OBM filtrate. These fluids have similar characteristics in terms of chemical composition, fluorescence, and absorbance properties. The LWD/MWD/Wire-Line logs are influenced by this mix, but typically without clearly identifying the fluids produced or tested, making it difficult to calculate reserves and the prospects in the formation.

The methodology used herein includes chemical tracers of different physicochemical characteristics, with the purpose of differentiating the filtrate of the OBMs from the hydrocarbons present in the formation using the Wireline/LWD/MWD tools, particularly those which measure radiation absorbance in-situ in real time. The method improves the identification of the hydrocarbon in the reservoir as compared with the filtrate of the OBM during the taking of electrical logs in the drilling and production phase. The method allows the determination of prospective hydrocarbon-producing zones without the uncertainty of which fluids are being measured, for instance the uncertainly of measuring fluids contaminated by leaking OBM filtrate.

The method differentiates between the presence of the OBM, whether the OBM itself or OBM filtrate, and hydrocarbons produced from the formation, e.g., crude oil. In one non-limiting embodiment, the crude oil is “light crude oil”, which is defined as having a low density and flows freely at room temperature; it has a low viscosity, low specific gravity, and high API gravity due to the presence of light hydrocarbon fractions. In another non-restrictive version, the light crude oil has an API density of 30 API or higher.

It will be understood that the baseline absorbance is determined for OBM filtrate rather than OBM itself because OBMs are too thick to obtain absorbance measurements.

In one non-limiting embodiment, the tracers are chemical dyes. Specific examples of suitable dyes include, but are not necessarily limited to, chromophores which in turn are made of naphthalenes, aniline, nitrobenzene, aminophenols, azo-red, and the like; and also heavy solvent naphthas, yellow dyes, orange dyes, green dyes, and the like. The chemical tracer should have an absorbance spectrum different from the crude oil, in one non-limiting embodiment.

In another non-restrictive version, the tracer is a plurality of quantum dots. Quantum dots are semiconductor particles of a few nanometers in size, which have optical and electronic properties that differ from larger particles of the same material as a result of quantum mechanics. When quantum dots are illuminated by UV light, an electron in the quantum dot can be excited to a state of higher energy. The excited electron can drop back to a lower state releasing its energy as light, which is detectable. In brief, the quantum dots are fluorescent. They may be introduced into the OBM dispersed in a solvent, although it will be appreciated that because the quantum dots are not soluble in the solvent, a more precise term would be carrier fluid.

In a different non-limiting embodiment, the tracer may be a plurality of nanoparticles. As defined herein, nanoparticles are particles of matter that are between about 1 independently to about 100 nanometers in diameter; alternatively from about 2 independently to about 10 nanometers. Suitable nanoparticles include, but are not necessarily limited to, In one non-limiting embodiment, at least a portion of the surface of the quantum dots contain functional groups such as carboxyl groups, hydroxyl groups, and/or ether groups. In a different non-restrictive version, the quantum dots are doped with one or more of nitrogen, boron, silicon, and/or phosphorus. Further, in a different non-limiting embodiment, the quantum dots have at least a portion of the surface thereof which are hydrophilic and/or oleophilic. Again, it will be appreciated that because the nanoparticles are not soluble in a solvent, a more precise term would be carrier fluid. It should also be understood that the quantum dots and the nanoparticles would be filtered through with the OBM and be present in the OBM filtrate.

In a different, non-restrictive version, the tracer has an absorbance spectrum that oscillates between about 100 to about 2000 nm. In one non-limiting embodiment, the absorbance spectrum is determined by the crude oil whose presence is sought to be determined. In another non-limiting embodiment, the absorbance spectrum produced by the Wireline/MWD/LWD tool with optical sensors is within a range that oscillates between about 0 and about 4 Absorbance Units (AU).

The tracer can be delivered into the OBM by using any suitable solvent, but can also be introduced into the OBM without a solvent. Suitable solvents for introducing the chemical tracer or dye into the OBM include, but are not necessarily limited to, aromatic heavy solvent naphtha distillate, and/or fuel oil.

In one non-limiting embodiment, the amount of chemical tracer in the OBM ranges from about 10 ppm independently to about 2500 ppm, alternatively from about 100 ppm independently to about 2500 ppm. In one non-limiting embodiment, these are the minimum amounts of chemical tracer needed to obtain an absorbance signal. As used herein with respect to a range, the word “independently” means that any endpoint may be used together with any other endpoint to give a suitable alternative range.

The proportion of tracer in the OBM can range from about 0.01 vol % independently to about 2.5 vol % based on the total OBM volume; alternatively from about 0.05 vol % independently to about 2 vol %.

Because the OBM introduced into the hydrocarbon reservoir wellbore contains these tracers, and the crude oil flowing from the formation do not, they allow identifying mixtures of hydrocarbons with the filtrate of the OBM (or the OBM itself) because of different concentrations and other changes in the physical properties of the fluid sample that enters the electrical tool.

It should be noted that the chemical tracers used do not affect the properties of the OBM drilling fluid. There are no changes in the rheological properties or changes in the HPHT filtration properties that indicate any incompatibility with the chemistry of the drilling fluid. To reiterate, the logging tool looks for changes in the absorbance spectrum caused by the presence of the hydrocarbon (e.g., crude oil) present that is mixed with the OBM filtrate or OBM itself upon production of a sample through the tool.

The OBM may be a drilling fluid, drill-in fluid, a completion fluid, or the like. In one non-restrictive embodiment, the fluid is a drilling fluid or drill-in fluid.

The method described herein may be used together with a variety of wellbore operations including, but not necessarily limited to, drilling the well, completing the well, and combinations thereof. It will be understood that the data, e.g., determining absorbance values and comparing absorbance values, are collected in real time as the well is drilled or completed.

The method and compositions described herein will now be discussed with reference to certain specific Examples which are intended to further illustrate, but not necessarily limit the method and the compositions.

### EXAMPLES

Tests #1 through 3 measured absorbance values with an In-situ Fluids eXplorer (IFX) Service wireline tool from Baker Hughes that can measure Gas Oil Ratio (GOR), absorbance, fluorescence, density, viscosity, and refractive index. Absorbance values were measured for a Colombian crude oil mixed with Fuel Oil #4 as a solvent to deliver a green tracer dye at three concentrations: 25 vol. %, 50 vol. %, and 75 vol. %. Although a Colombian crude oil was used in these Examples, it will be appreciated that the method described herein is equally applicable to other light crude oils.

Tests #4 through 9 measured absorbance values with the IFX tool for two samples: Tests #4 through #6 were of the Colombian crude oil with OBM green dye tracer filtrate at 2500 ppm at concentrations of 25 vol. %, 50 vol. %, and 75 vol. %, respectively. Tests #7 through #9 were of the Colombian crude oil with OBM red dye tracer filtrate at 2500 ppm at concentrations of 25 vol. %, 50 vol. %, and 75 vol. %, respectively. It will be appreciated that for the laboratory tests in these Examples, the 2500 ppm is based on the volume of the base oil, which was then used to make the OBM for a total volume of 1.4 L.

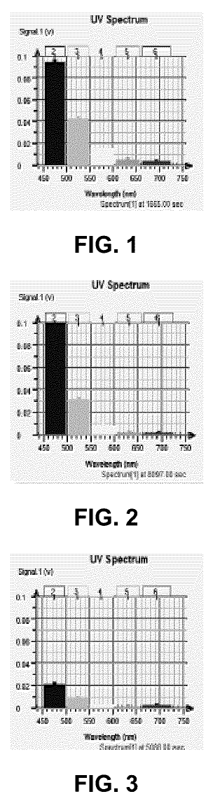

For the purposes of the absorbance values for the UV Spectrum in each of FIGS. 1-13, the colors have the wavelengths of Table I

TABLE I
Color Wavelengths

| Color | Wavelength, nm |
|---|---|
| Red | 535 |
| Orange | 465 |
| Green | 593 |
| Blue | 549 |

FIG. 1 is a chart of the UV base line absorbance spectrum for the Colombian crude oil used in all Tests. FIG. 2 is a chart of the UV base line absorbance spectrum for Fuel Oil #4. It can be seen that the spectrums presented in FIGS. 1 and 2 are very similar, which illustrates the difficulty of distinguishing between hydrocarbons.

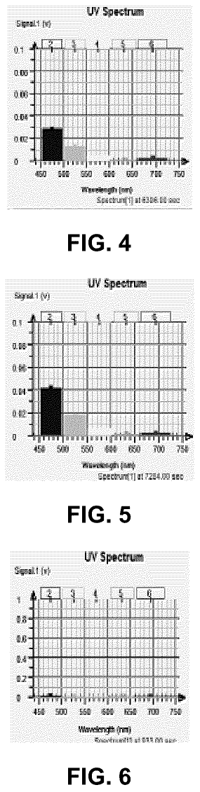

FIG. 3 is a chart of the UV absorbance spectrum for the Colombian crude oil with Fuel Oil #4, in a ratio of 25 vol. % to 75 vol. %, and 2500 ppm green dye (Test #1). FIG. 4 is a chart of the UV absorbance spectrum for the Colombian crude oil with Fuel Oil #4, in a ratio of 50 vol. % to 50 vol. %, and 2500 ppm green dye (Test #2). FIG. 5 is a chart of the UV absorbance spectrum for the Colombian crude oil with Fuel Oil #4, in a ratio of 75 vol. % to 25 vol. %, and 2500 ppm green dye (Test #3). It may be seen that the greater the amount of crude oil, the more the absorbance spectra resemble the base line absorbance spectrum of FIG. 1. Thus, these spectra indicate the ability to detect crude oil in a mixture of crude oil and fuel oil.

FIG. 6 is a chart of the UV base line absorbance spectrum for 100 vol. % filtrate with 2500 green dye; it can be seen that absorbance is very low.

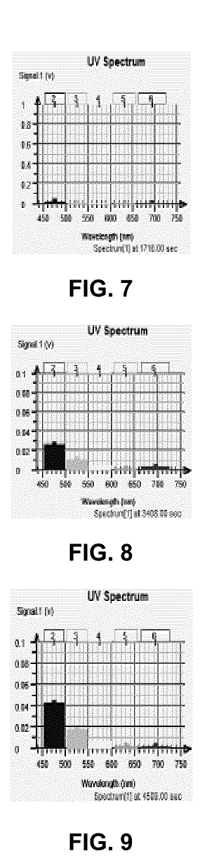

FIG. 7 is a chart of the UV absorbance spectrum for the Colombian crude oil and filtrate, in a ratio of 25 vol. % to 75 vol. %, with 2500 ppm green dye (Test #4). FIG. 8 is a chart of the UV absorbance spectrum for the Colombian crude oil and filtrate, in a ratio of 50 vol. % to 50 vol. %, with 2500 ppm green dye (Test #5). FIG. 9 is a chart of the UV absorbance spectrum for the Colombian crude oil and filtrate, in a ratio of 75 vol. % to 25 vol. %, with 2500 ppm green dye (Test #6). Thus, these tests indicate that the amount of filtrate can be detected since the absorbance value measured where there is more filtrate, the more the absorbance value looks like the filtrate base line spectrum of FIG. 6.

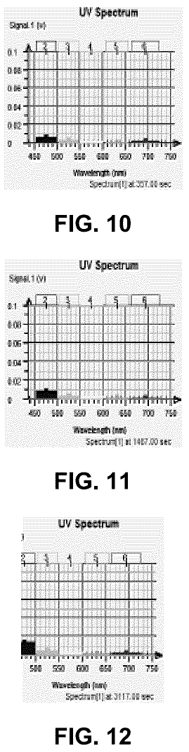

FIG. 10 is a chart of the UV base line absorbance spectrum for 100 vol. % filtrate with 2500 ppm red dye in place of the green dye. Again, it can be seen that absorbance is very low.

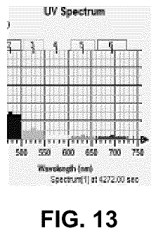

FIG. 11 is a chart of the UV absorbance spectrum for the Colombian crude oil with filtrate, in a ratio of 25 vol. % to 75 vol. %, with 2500 ppm red dye (Test #7). FIG. 12 is a chart of the UV absorbance spectrum for the Colombian crude oil with filtrate, in a ratio of 50 vol. % to 50 vol. %, with 2500 ppm red dye (Test #8). FIG. 13 is a chart of the UV absorbance spectrum for the Colombian crude oil with filtrate, in a ratio of 75 vol. % to 25 vol. %, with 2500 ppm red dye (Test #9). Once again, these tests indicate that the amount of filtrate can be detected where the absorbance value measured reflects the more filtrate, the more the absorbance value looks like the filtrate base line spectrum of FIG. 10.

In the foregoing specification, the method has been described with reference to specific embodiments thereof and has been shown as effective in providing a method for determining the amount of a hydrocarbon containing a tracer when mixed with another hydrocarbon, where both of the hydrocarbons have similar absorbance spectra.

It will be evident that various modifications and changes can be made to the methods and compositions described herein without departing from the broader scope of the invention as set forth in the appended claims. Accordingly, the specification is to be regarded in an illustrative rather than a restrictive sense. For example, specific OBMs, solvents, chemical tracers, dyes, tools, ratios, and proportions thereof falling within the claimed parameters, but not specifically identified or tried in a particular method or composition to improve the determination of crude oil within an OBM herein, are expected to be within the scope of this application.

The present application may suitably comprise, consist, or consist essentially of the elements disclosed and may be practiced in the absence of an element not disclosed. For instance, the method for determining whether crude oil has been produced from a hydrocarbon reservoir wellbore that has been drilled using an OBM, where the method may comprise, consist essentially of, or consist of, providing the OBM characterized in that the OBM comprises a tracer, filtering the OBM to obtain an OBM filtrate comprising the tracer, determining a baseline absorbance value for the OBM filtrate containing the tracer, determining a baseline absorbance value for the crude oil, introducing the OBM filtrate into a hydrocarbon reservoir wellbore, conducting a wellbore operation with the OBM, producing a sample from the well , measuring a measured absorbance value of the sample, and comparing the measured absorbance value with the baseline absorbance values for the OBM filtrate and the baseline absorbance value for the crude oil to determine whether crude oil is present in the sample.

There may be further provided an oil-based mud (OBM) composition comprising, consisting essentially of, or consisting of an oil-based mud, and a tracer.

Additionally, there may be provided a sample composition that comprises, consists essentially of, or consists of crude oil produced from a hydrocarbon reservoir, an OBM filtrate, and a tracer.

As used herein, the terms “comprising,” “including,” “containing,” “characterized by,” and grammatical equivalents thereof are inclusive or open-ended terms that do not exclude additional, unrecited elements or method acts, but also include the more restrictive terms “consisting of” and “consisting essentially of” and grammatical equivalents thereof. As used herein, the term “may” with respect to a material, structure, feature, or method act indicates that such is contemplated for use in implementation of an embodiment of the disclosure and such term is used in preference to the more restrictive term “is” so as to avoid any implication that other, compatible materials, structures, features, and methods usable in combination therewith should or must be, excluded.

As used herein, the singular forms “a,” “an,” and “the” are intended to include the plural forms as well, unless the context clearly indicates otherwise.

As used herein, the term “and/or” includes any and all combinations of one or more of the associated listed items.

As used herein, relational terms, such as “first,” “second,” “top,” “bottom,” “upper,” “lower,” “over,” “under,” etc., are used for clarity and convenience in understanding the disclosure and do not connote or depend on any specific preference, orientation, or order, except where the context clearly indicates otherwise.

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” in reference to a given parameter is inclusive of the stated value and has the meaning dictated by the context (e.g., it includes the degree of error associated with measurement of the given parameter).

What is claimed is:

1. A method for determining whether crude oil has been produced from a hydrocarbon reservoir wellbore that has been drilled using an oil-based mud (OBM), the method comprising:
providing the OBM characterized in that the OBM comprises a tracer;
filtering the OBM to obtain an OBM filtrate comprising the tracer;
determining a baseline absorbance value for the OBM filtrate containing the tracer;
determining a baseline absorbance value for the crude oil;
introducing the OBM filtrate into a hydrocarbon reservoir wellbore;
conducting a wellbore operation with the OBM;
producing a sample from the well
measuring a measured absorbance value of the sample, and
comparing the measured absorbance value with the baseline absorbance values for the OBM filtrate and the baseline absorbance value for the crude oil to determine whether crude oil is present in the sample.

2. The method of claim 1, where the tracer is selected from the group consisting of
a plurality of quantum dots;
a plurality of nanoparticles;
a dye having an absorbance spectrum different from the crude oil and the OBM filtrate, and combinations thereof; and
combinations of these tracers.

3. The method of claim 1, where the sample comprises:
crude oil produced from a hydrocarbon reservoir,
an oil-based mud (OBM) filtrate; and
a tracer; and where
the sample has an absorbance spectrum that oscillates between about 100 to about 2000 nm.

4. The method of claim 2, where the crude oil is light crude oil having an API density of 30 API or higher.

5. The method of claim 1, further comprises introducing the tracer into the OBM within a solvent, where the solvent is selected from the group consisting of aromatic heavy solvent naphtha distillate, fuel oil, and combinations thereof.

6. The method of claim 1, where the proportion of tracer in the OBM ranges from about 0.01 vol % to about 2.5 vol % of the total OBM.

7. The method of claim 1, where the wellbore operation is selected from the group consisting of drilling the well, completing the well, and combinations thereof, and where determining absorbance values and comparing absorbance values occur in real time while drilling and/or completing the well.

8. The method of claim 1, where the measuring the measured absorbance value of the sample is performed downhole.

9. The method of claim 8, where measuring the measured absorbance value of the sample is conducted using a logging tool selected from the group consisting of a wireline tool, a measurement while drilling (MWD) tool, a logging while drilling (LWD) tool, and combinations of these.

10. An oil-based mud (OBM) composition comprising:
an oil-based mud filtrate; and
a tracer.

11. The OBM composition of claim 10, where the tracer is selected from the group consisting of
a plurality of quantum dots;
a plurality of nanoparticles;
a dye having an absorbance spectrum different from the crude oil, the OBM filtrate, and combinations thereof; and
combinations of these tracers.

12. A sample composition comprising:
crude oil produced from a hydrocarbon reservoir,
an oil-based mud (OBM) filtrate; and
a tracer.

13. The sample composition of claim 12, where the tracer is selected from the group consisting of
a plurality of quantum dots;
a plurality of nanoparticles;
a dye having an absorbance spectrum different from the crude oil, the OBM filtrate, and combinations thereof; and
combinations of these tracers.

14. The sample composition of claim 13, where the sample has an absorbance spectrum that oscillates between about 100 to about 2000 nm.

15. The sample composition of claim 12, where the crude oil is light crude oil having an API density of 30 API or higher.

16. The sample composition of claim 12, where the tracer is a chemical tracer and the sample composition further comprises a solvent selected from the group consisting of aromatic heavy solvent naphtha distillate, fuel oil, and combinations thereof, where the chemical tracer was dissolved in the solvent.

17. The sample composition of claim 12, where the proportion of tracer in the OBM ranges from about 0.01 vol % to about 2.5 vol % of the OBM.
